(19) United States
(12) Patent Application Publication
(10) Pub. No.: US 2024/0001001 A1
(43) Pub. Date: Jan. 4, 2024

(54) COMPOSITIONS AND METHODS FOR SOFT TISSUE AUGMENTATION18348273

(71) Applicant: Athena Devices, Inc., Decatur, GA (US)

(72) Inventors: David N. Ku, Decatur, GA (US); Christopher A. Bresette, Atlanta, GA (US)

(73) Assignee: Athena Devices, Inc., Decatur, GA (US)

(21) Appl. No.: 18/351,305

(22) Filed: Jul. 12, 2023

Related U.S. Application Data
(63) Continuation of application No. PCT/US22/12455, filed on Jan. 14, 2022.
(60) Provisional application No. 63/138,267, filed on Jan. 15, 2021.

(51) Int. Cl. A61L 27/50; A61L 27/54; A61L 27/52; A61L 27/18

(52) U.S. Cl. CPC A61L 27/502; A61L 27/54; A61L 27/52; A61L 27/18; A61L 2400/06; A61L 2430/34

(57) ABSTRACT

A tissue-filling composition comprises an injectable material having the properties of a Bingham plastic. The tissue-filling composition is a solid which partially liquefies as it is injected through a needle or cannula into solid tissue. The tissue-filling composition may be a poly(vinyl alcohol) produced from an aqueous solution which is subjected to a single freeze-thaw cycle under conditions which cause the poly(vinyl alcohol) to have the properties of a Bingham plastic.

### CROSS REFERENCE TO RELATED APPLICATION

This application is a continuation of PCT Application No. PCT/US22/12455 (Attorney Docket No. 59969-703601), filed Jan. 14 2022, which claims the benefit of U.S. Provisional Application No. 63/138,267 (Attorney Docket No. 59969-703.101), filed on Jan. 15, 2021, the full disclosure of which is incorporated herein by reference.

### BACKGROUND OF THE INVENTION

### 1. Field of the Invention

The present invention relates generally to compositions and devices for tissue filling and augmentation and methods for their manufacture and use. More particularly, the present invention relates to tissue-filling compositions and devices intended for facial tissue injection.

Solid and liquid tissue fillers are known. Solid tissue fillers, often formed as implants, have the advantage of being stable and retaining their shapes but are not injectable and can be difficult to initially size, requiring that an implantation site be carefully formed.

Liquid fillers, in contrast, can be injectable and easier to place as they readily conform to an injection site, and the injection site typically requires lass preparation. After injection, however, the liquid materials can be less stable, lose their shape, and move to unintended locations.

Both solid and liquid tissue fillers often fail to match the stiffness or hardness of a tissue being filled. An implant which is either harder or softer than the surrounding tissue can result in unnatural feel, particularly when a softer material is implanted over a bony structure, such as in a patient's face. More seriously, an implant which is harder than the surrounding tissue can cause tissue erosion which can be a significant clinical problem.

For these reasons, it would be desirable to provide additional, alternative, and improved compositions for tissue filling and augmentation and methods for their manufacture and use. In particular, it would be desirable to have tissue filling compositions which can be delivered with the ease of an injectable material while displaying the characteristic of a solid implant after injection. It would be further desirable to provide tissue-filling compositions which can more closely match the stiffness of a tissue being filled. At least some of these objectives will be met by the inventions described below.

### 2. Listing of Background Art

Relevant patents and publications include U.S. Pat. Nos. 5,981,826; 5,941,909; 5,876,447; 10,821,277; 10,660,762; US2007/0212385; US2009/0069739; US2017/0304039; US2020/0188163.

### SUMMARY OF THE INVENTION

In a first aspect, the present invention provides a composition of matter comprising a Bingham plastic in a form suitable for implanting into mammalian tissue. Typically, the Bingham plastic is a viscoplastic material that behaves as a rigid body at low shear stresses but flows as a viscous fluid at high shear stresses. Such compositions may be produced by a freeze-thaw process as described in more detail below.

In many instances, the composition of matter may be present in a container and be configured to be extruded from the container into solid tissue. In other instances, the composition of matter may be pre-formed into a shape suitable for surgical implantation into solid tissue.

In preferred embodiments, the composition of matter comprises a poly(vinyl alcohol) having a molecular weight in a range from 8 kDa to 200 kDa, often from 85 kDa to 186 kDa, usually from 146 kDa to 186 kDa. The poly(vinyl alcohol) may have an average degree of hydrolysis 80% to 100%, often from 87% to 99.9%, and usually from 99% to 99.9%.

In specific instances, the poly(vinyl alcohol) is present in an aqueous solution and subjected to a single freeze-thaw cycle under conditions which cause the poly(vinyl alcohol) to have the properties of a Bingham plastic.

The composition of the present invention may further comprise a bioactive agent, such as protein, heparin, fibronectin, collagen, a sugar, a βAPN, an antibody, a cytokine, an integrin, a protease, a matrix inhibitor, an anticoagulant, a sphyngolipid, a thrombin, a thrombin inhibitor, a glycosaminoglycan, a topical anesthetic, and the like.

In a second aspect, the present invention provides a method for producing a composition suitable for soft tissue implantation. The method comprises freezing an aqueous solution of a poly(vinyl alcohol) in a container at a temperature of 0° C. or below to produce a solid poly(vinyl alcohol) having a shape determined by an interior shape of the container. After freezing, the temperature of the poly(vinyl alcohol) solid is raised to 1° C. or above, typically to room temperature, causing the poly(vinyl alcohol) to become a viscoplastic material that behaves as a rigid body at low stresses but flows as a viscous fluid at high stress. Optionally, the solid poly(vinyl alcohol) material may be stored or otherwise held in its frozen state prior to warming for extended times of days, weeks, or months prior to raising the temperature. Freezing of the solid poly(vinyl alcohol) material after it has been formed with the Bingham plastic properties is generally not suitable as it will reverse or eliminate the Bingham plastic properties.

The solid poly(vinyl alcohol) materials produced by these methods can be extruded from the container into solid tissue. Alternatively, the solid poly(vinyl alcohol) materials produced by these methods can be surgically implanted into solid tissue.

The poly(vinyl alcohols) used in the methods of the present invention typically have a molecular weight in a range from 8 kDa to 200 kDa, often from 85 kDa to 186 kDa, and usually from 146 kDa to 186 kDa. The poly(vinyl alcohol) typically has an average degree of hydrolysis in a range from 80% to 100%, often from 87% to 99.9%, and usually from 99% to 99.9%. The poly(vinyl alcohol) is usually frozen at a temperature in a range from 0° C. to −10° C. for a time sufficient to freeze the initial liquid solution, in the range from 10 minutes to 48 hours.

In a third aspect, the present invention provides product produced by the processes just described. Those products may further comprise a bioactive agent, such as a protein, heparin, fibronectin, collagen, a sugar, a βAPN, an antibody, a cytokine, an integrin, a protease, a matrix inhibitor, an anticoagulant, a sphyngolipid, a thrombin, a thrombin inhibitor, a glycosaminoglycan, and a topical anesthetic.

In a fourth aspect, the present invention provides a method for augmenting tissue in a patient. The method comprises providing a solid implantation material having the properties of a Bingham plastic and injecting the solid implantation material through a lumen of a tubular body into soft tissue, wherein passage of the solid implantation material through said lumen deforms and applies a shear stress on the solid implantation material which causes at least an outer portion of the solid implantation material to liquefy, wherein the liquefied portion of the solid implantation material re-solidifies after implantation in the tissue.

In particular instances, the solid implantation material is injected through a needle or cannula into the target tissue, often being injected manually using a syringe on a needle. Suitable target tissues include any soft tissue, including but not limited to a patient's face, vocal cord, buttock, calf, and breast tissue. In specific instances the solid implantation material may be injected over a region of bone, often being injected into tissue on the patient's face.

In such tissue augmentation methods, the solid implantation material typically comprises a Bingham plastic having viscoplastic properties which behaves as a rigid body at low stresses such as after implantation but flows as a viscous fluid under high stresses such as during injection. Suitable Bingham plastic materials may be produced by a freeze-thaw process, as described below.

Exemplary implantation materials of the present invention may comprise a poly(vinyl alcohol) having a molecular weight in a range from 8 kDa to 200 kDa, often from 85 kDa to 186 kDa, and usually from 146 kDa to 186 kDa. The poly(vinyl alcohol) typically has an average degree of hydrolysis in a range from 80% to 100%, often from 87% to 99.9%, and usually from 99% to 99.9%. Often, the poly(vinyl alcohol) solid is produced by subjecting an aqueous poly(vinyl alcohol) solution to one or more freeze-thaw cycle under conditions which cause the resulting solid poly(vinyl alcohol) material to have the properties of a Bingham plastic.

For most aqueous poly(vinyl alcohol) solutions, a single freeze-thaw cycle is sufficient to solidify and impart the desired Bingham plastic properties. For lower molecular weight poly(vinyl alcohol), e.g. below 146 kDa, more often below 85 kDa, and/or, more dilute aqueous starting solutions of poly(vinyl alcohol), e.g. below 2.5% by weight, more often below 1% by weight, however, two or more freeze-thaw cycles may be required to achieve the desired Bingham plastic properties. Any particular combination of molecular weight and weight percent can of course be tested to see if the desired Bingham properties are achieved before adopting those values for production.

Other suitable implantation materials which may be converted into Bingham polymers include but are not limited to polyethylene glycols (PEG's) typically having a molecular weight in a range from 400 D to 20 kD, poly(glycolic acid) (PGA), Dextran solutions, and other water-soluble long chain polymers.

The Bingham plastic tissue augmentation materials of the present invention may be prepared and stored in various ways. For example, the frozen poly(vinyl alcohol) or other hydrogel may be thawed after being frozen one time and then be stored without refreezing until use, typically at room temperature. Alternatively, the frozen poly(vinyl alcohol) or other hydrogel may be stored without thawing until use, i.e., initially frozen and stored while frozen until ready to be thawed prior to use. As one freeze-thaw cycle is the preferred preparation protocol for many formulations, the temperature of the stored frozen formulations should be tracked to assure that the formulations do not accidentally thaw during storage due to refrigeration failure or other causes. It is preferred that the poly(vinyl alcohol) hydrogel formulations of the present invention be frozen only once prior to thawing and implantation into a patient.

In a fifth aspect, the present invention provides an article for delivering a composition suitable for soft tissue implantation into a target tissue site. Such articles comprise a container having an interior and a composition of matter, as previously described, present in the interior of the container. The composition of matter typically fills and conforms to the interior of the container, and in some instances, the article may further comprise an injection element fluidly coupled to the container and having a cross-sectional dimension smaller than a cross-sectional dimension of the container.

In exemplary embodiments, the container may have a cylindrical interior and the injection element may comprise a cylindrical needle a one end of the container. In such instances, the article may comprise a needle and syringe assembly having a plunger configured to manually extrude the composition of matter from the interior of the container. Often, the composition of matter is at least partially liquefied as it passes from the interior of the container though a lumen of the needle, and often the at least partially liquefied composition of matter solidifies after being released from a distal end of the needle into tissue.

The implantation materials of the present invention will preferably be elastic and have a stiffness or harness matching the stiffness or hardness of the tissue that is being augmented. In exemplary cases, the implantation materials of the present invention will have a compressive modulus of elasticity in a range from 1 kPa to 5 MPa, preferably from 10 kPa to 500 kPa, and even more preferably 50 kPa to 200 kPa. Specific values within these ranges may be selected to match those of particular target tissues. The compressive modulus of elasticity is defined as the ratio of mechanical stress to strain in an elastic material when that material is being compressed, expressed as the compressive force per unit area/change in volume per unit volume. The compressive modulus of elasticity, also referred to as the elastic modulus E, of the implantation materials of the present invention may be measured by known techniques. See, e.g.: Dowling, Mechanical Behavior of Materials: Engineering Methods for Deformation, Fracture, and Fatigue—2nd edition 1999. Prentice-Hall; Chapter 4—Mechanical testing: Tension test and Other Basic Tests. Section 4.6 Compression Test, 4.6.1 Test Methods for Compression, 4.6.2 Material Properties in Compression, Pages 135-139.

### BRIEF DESCRIPTION OF THE DRAWINGS

The novel features of the invention are set forth with particularity in the appended claims. A better understanding of the features and advantages of the present invention will be obtained by reference to the following drawings and detailed written description that set forth illustrative embodiments in which the principles of the invention are utilized.

FIG. 1 is a chart summarizing steps suitable for preparing the solid implant materials of the present invention.

FIG. 2 illustrates a first exemplary container suitable for preparing the solid implant materials of the present invention.

FIG. 3 illustrates a second exemplary container with portions broken away suitable for preparing and injecting the solid implant materials of the present invention.

FIG. 4. illustrates use of the container of FIG. 3 for injecting the solid implant into a target site on a patient's face.

### DETAILED DESCRIPTION OF THE INVENTION

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

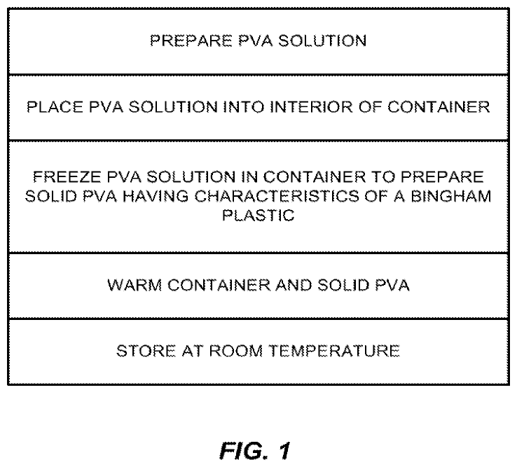

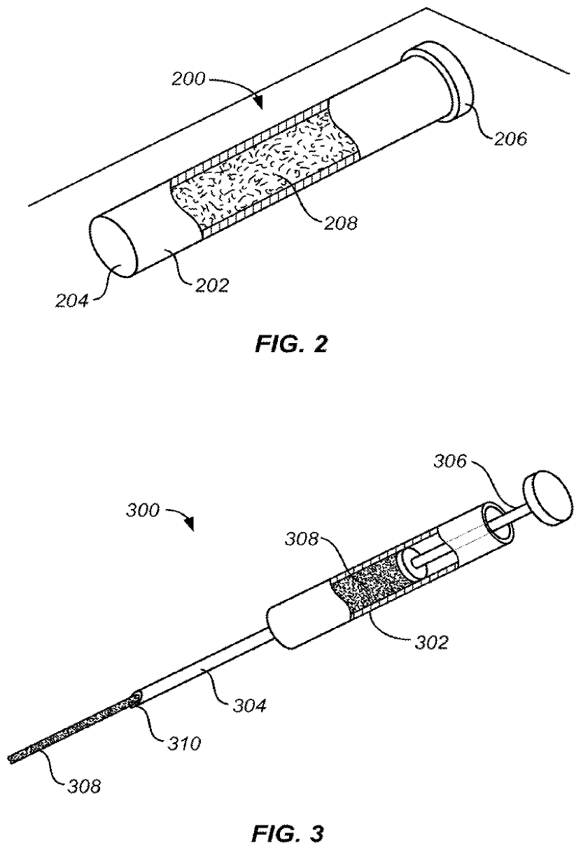

Referring to FIG. 1, poly(vinyl alcohol) (PVA) solid implants according to the present invention may be prepared from a PVA hydrogel by placing the hydrogel inside a container, such as a cylindrical container 200, as shown in FIG. 2 or a syringe barrel 302, as shown in FIG. 3. Suitable PVA hydrogels may be prepared as described in Example 1 or Example 4, below.

The container is typically filled with the hydrogel so that an outer surface of the hydrogel conforms to an interior surface of the container. Both container 200 and syringe barrel 302 are illustrated as cylinders, but it will be appreciated that at least the container 200 may have a variety of shapes and can act as a mold to prepare an implant having a desired shape. Such shaped implants would typically be used for surgical implantation without extrusion and liquefication. In most instances, however, the solid implants of the present invention will be intended for delivery by extrusion through a needle or cannula, as with the syringe embodiment of FIG. 3, so that the implant will at least partially liquefy as it passes through the cannula or needle, and then re-solidify as it is deposited at a target tissue location.

The container 200 as shown in FIG. 2 may have a cylindrical body 202 with a closed end 204 and an open end with a removable cap 206. The PVA hydrogel 208 may be poured or otherwise introduced into an interior of the cylindrical body 202, and the cap 206 placed over the open end. The container may then be placed in a freezer as discussed in Examples 1 and 4, below. Exposing the container with the PVA hydrogel to a single freeze thaw cycle under the conditions described in Examples 1 and 4 prior to warming solidifies the PVA with the resulting PVA solid having the properties of a Bingham solid, i.e., an exterior portion of the PVA solid will at least partially liquefy as the solid is subjected to a shear stress, such as by extrusion through a needle, cannula or other lumen having a cross-sectional area less than that of the PVA solid prior to extrusion.

The syringe container 300 of FIG. 3 includes the syringe barrel 302, a needle 304, and a plunger 306. The PVA solid initially in the interior of the syringe barrel 302 is extruded through the needle 304 by manually depressing plunger 306, causing a cylindrical flow 308 of the PVA to exit a distal tip 310 of the needle. The PVA is a solid when present in the interior of the syringe barrel 302 and at least partially liquefies as it passes into the lumen of the needle due to the stresses caused by passing from the large diameter syringe barrel into the smaller diameter needle. As the at least partially liquefied PVA passes from the distal tip 310 of the needle 304, the PVA quickly re-solidifies in flow 308 having an exterior shape determined by the cross-sectional shape of the needle tip 310.

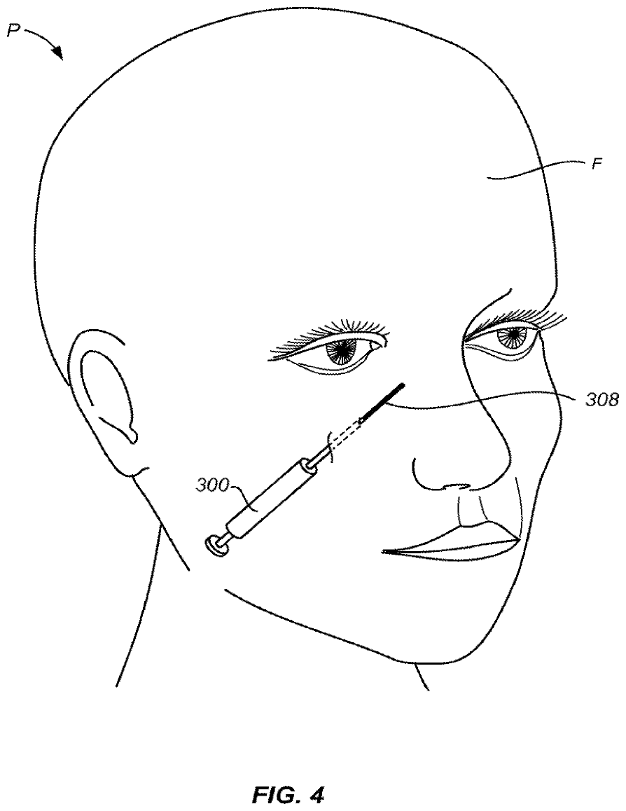

As shown in FIG. 4, the solid PVA implants of the present invention can be injected to target locations on a patient's P face F. The solid PVA implants are particularly useful for injection beneath superficial tissue and over a facial bone of the patient, for example beneath the patient's eye as illustrated.

Example 1: A poly(vinyl alcohol) (PVA) solid in accordance with the principles of the present invention is made from a (PVA) hydrogel formed by dissolving a PVA powder in water. The PVA powder has a molecular weight in a range 9,000 to 186,000, preferably from 146,000-186,000, and is hydrolyzed above 80% hydrolyzed, preferably above 99%. Such PVA powders are commercially available from suppliers such as Sigma-Aldrich, Celanese, Kuraray, and Sekisui. The solution is placed in an interior of a container, and the container is placed in a freezer at a temperature in a range from −1° C. to −10° C. for a time sufficient to allow the PVA hydrogel to freeze solid, typically from 10 minutes to 48 hours for container volumes from 0.1 ml to 20 ml, often from 10 ml to 100 ml. The container carrying the solid PVA in its interior may then be allowed to warm to room temperature and may be stored at a temperature between 1° C. and 54° C. (33° F. to 130° F.), typically at room temperature. The solid PVA in the interior of the container is now ready for introduction into a tissue site in a patient's body tissue as a medical implant, either by injection or surgical implantation.

Example 2: The container in example 1 may comprise a 1 ml syringe, having a cylindrical barrel with a 5 mm diameter and a 65 mm length and a small gauge needle or cannula between 34 Gauge (0.0.51 mm I.D.) and 10 Gauge (2.693 mm I.D.), preferably between 30G (0.159 mm I.D.) and 21G (0.514 mm I.D.) cannulas. The solid PVA implants of the present invention undergo a partial liquification as they are injected from the cylindrical barrel through the small gauge needle or cannula, re-solidifying when released into the soft tissue after the stress of injection is relieved. The dimensions of the re-solidified PVA implants will be determined by the cross-sectional dimensions of the small gauge needle or cannula.

Example 3: Solid 5 mm-diameter cylindrical implants composed of silicone, polyurethane, polytetrafluoroethylene (PTFE), polyethylene are placed in the barrel of a syringe similar to that described in Example 2. When applying a force to the syringe plunger similar to that utilized in Example 2, it is found that these solid implants will not pass through a small gauge needle.

Example 4: A hydrogel is made by dissolving PVA having a molecular weight in a range from 146,000 to 186,000 and being hydrolyzed above 99% in an aqueous solvent. A mold having a 5 mm interior diameter is filled with the solution. The mold is frozen until the PVA material is a solid mass. The mold is allowed to warm, and the solid PVA material may be removed from the mold and inserted into a patient's body as a medical implant. For example, the solid PVA material can be placed through an introducer that has a smaller dimension than the molded solid PVA material having a 5 mm diameter exterior can be introduced through a cannula with an inside diameter of 0.3 mm or smaller because the molded solid PVA material acts as a Bingham plastic which can liquefy about its exterior when stressed as it is forced through the smaller cannula.

Example 5: PVA hydrogel is made by dissolving PVA powder having a molecular weight of 146 kDa to 186 kDa in water. The solution is placed in a syringe or mold. The mold is placed in a freezer for enough time to cause the device to entirely freeze. The PVA construct is removed, at least partially, from said mold and, while immersed in water, is re-frozen and thawed one or more times. The resultant construct is an elastic solid that requires a significant force to be pushed through a small gauge cannula (above 21Gauge). The solid PVA implant prepared in this manner does not act as a Bingham plastic and does not at least partially liquefy as a result of the stress being applied during the attempted injection.

Example 6: A polyurethane device made by methods well known in the art is molded into a cylinder. The cylindrical polyurethane device will not pass through a cannula having an inner lumen diameter which is 80% or less than the outer device diameter.

Example 7: A PVA solution of 10% by weight is made by dissolution of the PVA in saline. The solution is not subjected to freezing. The resultant product is not solid, but liquid. It has a zero-yield stress. The material deforms easily under its own weight and will not stay in the shape of the mold. Thus, it does not act as a Bingham plastic.

The foregoing embodiments are presented by way of example only; the scope of the present invention is to be defined by the following claims.

1. A composition of matter comprising a Bingham plastic in a form suitable for implanting into mammalian tissue.

2. A composition of matter as in claim 1, wherein the Bingham plastic is a viscoplastic material that behaves as a rigid body at a low shear stress but flows as a viscous fluid at a high shear stress produced by a freeze-thaw process.

3. A composition of matter as in claim 1, wherein the composition of matter is present in a container and configured to be extruded from the container into soft tissue.

4. A composition of matter as in claim 1, wherein the composition of matter is pre-formed into a shape suitable for surgical implantation into soft tissue.

5. A composition of matter as in claim 1, wherein the composition of matter comprises a poly(vinyl alcohol) hydrogel having a molecular weight in a range from 8 kDa to 200 kDa, often from 85 kDa to 186 kDa, usually from 146 kDa to 186 kDa subjected to multiple freeze-thaw cycles to form the Bingham plastic.

6. A composition of matter as in claim 5, wherein the poly(vinyl alcohol) hydrogel has been hydrolyzed in a range from 80% to 100%, often from 87% to 99.9%, and usually from 99% to 99.9% prior to forming the Bingham plastic.

7. A composition of matter as in claim 5, wherein the poly(vinyl alcohol) hydrogel is present in an aqueous solution and subjected to the freeze-thaw cycle under conditions which cause the poly(vinyl alcohol) to have the properties of a Bingham plastic.

8. The composition of claim 1, further comprising a bioactive agent.

9. (canceled)

10. A method for producing a composition suitable for soft tissue implantation, said method comprising:
freezing an aqueous solution of a poly(vinyl alcohol) in a container at a temperature of 0°C. or below to produce a poly(vinyl alcohol) solid having a shape determined by an interior shape of the container;
raising a temperature of the poly(vinyl alcohol) solid to 10° C. or above, wherein the poly(vinyl alcohol) becomes a viscoplastic material that behaves as a rigid body at low stresses but flows as a viscous fluid at high stress.

11. A method as in claim 10, wherein the solid poly(vinyl alcohol) solid is configured to be extruded from the container into solid tissue.

12. A method as in claim 10, wherein the solid poly(vinyl alcohol) solid is configured to be surgically implanted into solid tissue.

13. A method as in claim 10, wherein the poly(vinyl alcohol) solid has a molecular weight in a range from 8 k Da to 200 kDa, often from 85 kDa to 186 kDa, usually from 146 kDa to 186 kDa.

14. A method as in claim 13, wherein the poly(vinyl alcohol) hydrogel was hydrolyzed in a range from 80% to 100%, often from 87% to 99.9%, and usually from 99% to 99.9%, prior to forming the Bingham plastic.

15. A method as in claim 13, wherein the poly(vinyl alcohol) hydrogel is frozen at a temperature in a range from −1° C to −10° C. for a time of at least 10 minutes prior to thawing.

16. A method as in claim 15, wherein (i) the frozen poly(vinyl alcohol) hydrogel is thawed after being frozen one time and stored without refreezing until use or (ii) wherein the frozen poly(vinyl alcohol) hydrogel is stored without thawing until use or (iii) wherein the poly(vinyl alcohol) hydrogel is frozen only once prior to thawing and implantation into a patient.

17. (canceled)

18. (canceled)

19. A method as in claim 10, wherein the poly(vinyl alcohol) becomes a viscoplastic having a compressive modulus of elasticity in a range from 1 kPa to 5 MPa, preferably from 10 kPa to 500 kPa, and even more preferably 50 kPa to 200 kPa.

20. A composition of matter produced by the method of claim 10.

21. The composition of claim 20, further comprising a bioactive agent.

22. (canceled)

23. A method for augmenting tissue in a patient, said method comprising: providing a solid implantation material having the properties of a Bingham plastic; and
injecting the solid implantation material through a lumen of tubular body into solid tissue,
wherein passage of the solid implantation material through said lumen deforms and applies a shear stress on the solid implantation material which causes at least an outer portion of the solid implantation material to liquefy,
wherein the liquefied portion of the solid implantation material re-solidifies after implantation in the tissue.

24. The method of claim 23, wherein the solid implantation material is injected through a needle or cannula into the tissue.

25. The method of claim 24, wherein the solid implantation material is injected manually using a syringe on a needle or wherein the solid implantation material is injected over a region of bone.

26. (canceled)

27. The method of claim 23, wherein the solid implantation material is injected into tissue on the patient's face.

28. The method of claim 23, wherein the solid implantation material comprises a Bingham plastic produced by a freeze-thaw process, said Bingham plastic having viscoplastic properties and behaving as a rigid body at low stresses but flows as a viscous fluid at high stress.

29. A method as in claim 23, wherein the solid implantation material comprises a poly(vinyl alcohol) having a molecular weight in a range 8 k Da to 200 kDa, often from 85 kDa to 186 kDa, usually from 146 kDa to 186 kDa.

30. A method as in claim 29, wherein the poly(vinyl alcohol) is hydrolyzed in a range from 80% to 100%, often from 87% to 99.9%, and usually from 99% to 99.9%.

31. A method as in claim 29, wherein the poly(vinyl alcohol) is produced in an aqueous solution which is subjected to a freeze-thaw cycle under conditions which cause the poly(vinyl alcohol) to have the properties of a Bingham plastic.

32. An article for delivering a composition suitable for soft tissue implantation, said article comprising: a container having an interior; and a composition of matter as in claim 1 present in the interior of the container.

33. (canceled)

34. (canceled)

35. (canceled)

36. (canceled)
